(19) United States
(12) Patent Application Publication
(10) Pub. No.: US 2003/0171579 A1
(43) Pub. Date: Sep. 11, 2003

(54) Urethane derivatives

(76) Inventors: Werner Mederski, Erzhausen (DE); Horst Juraszyk, Seeheim-Jugenheim (DE); Dieter Dorsch, Ober-Ramstadt (DE); Christos Tsaklakidis, Weinheim (DE); Johannes Gleitz, Darmstadt (DE); Christopher Barnes, Bad Soden (DE)

Correspondence Address: Millen White Zelano & Branigan, Arlington Courthouse Plaza I, Suite 1400, 2200 Clarendon Blvd, Arlington, VA, 22201, US

(21) Appl. No.: 10/343,011

(22) PCT Filed: Jul. 13, 2001
(86) PCT No.: PCT/EP01/08130

(51) Int. Cl. C07D 279/12; C07D 271/02; C07D 271/08; C07D 285/04; C07C 275/40

(52) U.S. Cl. 544/59; 548/125; 558/417; 560/34; 564/60; 564/61; 548/253; 544/162; 544/400

(57) ABSTRACT

The invention relates to novel compounds of formula (I), wherein R, R¹ and R² have the meaning as cited claim 1. According to the invention, the compounds are inhibitors of the coagulation factors Xa and VIIa and can be used for treating thrombosis, myocardial infarct, arteriosclerosis, inflammation, apoplexy, angina pectoris, restenosis post-angioplasty, intermittent claudication, tumours, tumour related illnesses and/or tumour metastases.

The invention relates to compounds of the formula I

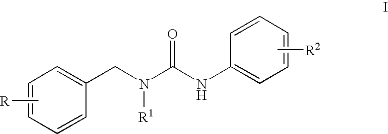

in which

R is CN, CH₂NH₂, —NH—C(═NH)—NH₂, —CO—N═C(NH₂)₂, —C(═NH)—NH₂, which may also be monosubstituted by Ar′, OH, O—COA, O—COAr, OCOOA, OCOO(CH₂)ₙN(A)₂, —COO(CH₂)ₙNA₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group

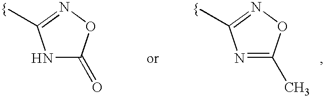

R¹ is R⁴, Ar, Ar′ or X,

R² is phenyl which is monosubstituted by SA, SOA, SO₂A, SONHA, SO₂NHA, CF₃, COOA, CH₂NHA, CN or OA,

R³ is CHal₃, OCOA or

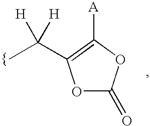

R⁴ is alkyl having 1-20 carbon atoms, in which one or two CH₂ groups may be replaced by O or S atoms and/or by —CH═CH— groups and/or 1-7 H atoms may be replaced by F,

A is H or alkyl having 1-20 carbon atoms,

A′ is alkyl having 1-10 carbon atoms,

Ar is phenyl or naphthyl, each of which is unsubstituted or mono-substituted, disubstituted or trisubstituted by A′, OH, OA′, NH₂, NHA′, NA′₂, NO₂, CF₃, CN, Hal, NHCOA, COOA, CONH₂, CONHA′, CONA′₂, SA, SOA, SO₂A, SO₂NH₂, SO₂NHA′ or SO₂NA′₂,

Ar′ is (CH₂)ₙ—Ar,

Het is a monocyclic or bicyclic saturated, unsaturated or aromatic heterocycle having 1 to 4 N, O and/or S atoms, which may be unsubstituted or monosubstituted, disubstituted or trisubstituted by A′, OA′, NH₂, NHA′, NA′₂, NO₂, CN, Hal, NHCOA′, NHSO₂A′, COOA, CONH₂, CONHA′, CONA′₂, COA, SO₂NH₂, SA′, SOA′, SO₂A′ and/or carbonyl oxygen,

X is (CH₂)ₙY,

Y is COOA or

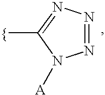

Hal is F, Cl, Br or I,

n is 1, 2, 3, 4, 5 or 6, and

m is 0 or 1,

and their pharmaceutically tolerated salts and solvates.

The invention also relates to the optically active forms, the racemates, the diastereomers and the hydrates and solvates, for example alcoholates, of these compounds.

The invention had the object of finding novel compounds having valuable properties, in particular those which can be used for the preparation of medicaments.

It has been found that the compounds of the formula I and their salts have very valuable pharmacological properties and are well tolerated. In particular, they exhibit factor Xa-inhibiting properties and can therefore be employed for combating and preventing thromboembolic illnesses, such as thrombosis, myocardial infarction, arteriosclerosis, inflammation, apoplexia, angina pectoris, restenosis after angioplasty and claudicatio intermittens.

The compounds of the formula I according to the invention may furthermore be inhibitors of the coagulation factors factor VIIa, factor IXa and thrombin in the blood coagulation cascade.

Aromatic amidine derivatives having an antithrombotic action are disclosed, for example, in EP 0 540 051 B1, WO 98/28269, WO 00/71508, WO 00/71511, WO 00/71493, WO 00/71507, WO 00/71509, WO 00/71512, WO 00/71515 and WO 00/71516. Cyclic guanidines for the treatment of thromboembolic illnesses are described, for example, in WO 97/08165. Aromatic heterocyclic compounds having factor Xa-inhibitory activity are disclosed, for example, in WO 96/10022. Substituted N-[(aminoiminomethyl)phenylalkyl)azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic illnesses. However, inhibition of thrombin may inhibit the fibrin formation involved in thrombus formation. The inhibition of thrombin can be measured, for example, by the method of G. F. Cousins et al. in Circulation 1996, 94, 1705-1712.

Inhibition of factor Xa can thus prevent the formation of thrombin.

The compounds of the formula I according to the invention and their salts engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example, by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour illnesses and/or tumour metastases. A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The compounds of the formula I can be employed as medicament active ingredients in human and veterinary medicine, in particular for the treatment and prevention of thromboembolic illnesses, such as thrombosis, myocardial infarction, arteriosclerosis, inflammation, apoplexia, angina pectoris, restenosis after angioplasty, claudicatio intermittens, venous thrombosis, pulmonary embolism, arterial thrombosis, myocardial ischaemia, unstable angina and strokes based on thrombosis. The compounds according to the invention are also employed for the treatment or prophylaxis of atherosclerotic diseases, such as coronary arterial disease, cerebral arterial disease or peripheral arterial disease. The compounds are also employed in combination with other thrombolytic agents in the case of myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis. The compounds are furthermore used in the cleaning of catheters and medical aids in vivo in patients, or as anticoagulants for the preservation of blood, plasma and other blood products in vitro. The compounds according to the invention are furthermore used for illnesses in which blood coagulation makes a crucial contribution to the course of the illness or represents a source of secondary pathology, such as, for example, in cancer, including metastasis, inflammatory disorders, including arthritis, and diabetes.

In the treatment of the illnesses described, the compounds according to the invention are also employed in combination with other thrombolytically active compounds, such as, for example, with “tissue plasminogen activator” t-PA, modified t-PA, streptokinase or urokinase. The compounds according to the invention are given either at the same time as or before or after the other substances mentioned.

Particular preference is given to simultaneous administration with aspirin in order to prevent recurrence of the clot formation.

The compounds according to the invention are also used in combination with blood platelet glycoprotein receptor (IIb/IIIa) antagonists, which inhibit blood platelet aggregation.

The invention relates to the compounds of the formula I and their salts and to a process for the preparation of the compounds of the formula I according to claim 1 and their salts, characterised in that

a) they are liberated from one of their functional derivatives by treatment with a solvolysing and/or hydrogenolysing agent by

i) liberating an amidino group from their oxadiazole derivative or oxazolidinone derivative by hydrogenolysis or solvolysis,

ii) replacing a conventional amino-protecting group with hydrogen by treatment with a solvolysing or hydrogenolysing agent or liberating an amino group protected by a conventional protecting group,

and/or

b) converting a base or acid of the formula I into one of its salts.

For all radicals which occur more than once, their meanings are independent of one another.

Above and below, the radicals and parameters R, R¹ and R² are as defined under the formula I, unless expressly stated otherwise.

The following abbreviations are used:

| | |
|---|---|
| Ac | acetyl |
| BOC | tert-butoxycarbonyl |
| CBZ or Z | benzyloxycarbonyl |
| DAPECI | N-(3-dimethylaminopropyl)-N-ethylcarbodiimide |
| DCCI | dicyclohexylcarbodiimide |
| DMF | dimethylformamide |
| dppf | 1,1′-bis(diphenylphosphine)ferrocene |
| Et | ethyl |
| Fmoc | 9-fluoroenylmethoxycarbonyl |
| HOBt | 1-hydroxybenzotriazole |
| Me | methyl |
| HONSu | N-hydroxysuccinimide |
| OBut | tert-butyl ester |
| Oct | octanoyl |
| OMe | methyl ester |
| OEt | ethyl ester |
| RT | room temperature |
| THF | tetrahydrofuran |
| TFA | trifluoroacetic acid |
| Trt | trityl (triphenylmethyl) |

The compounds of the formula I in which R is, for example, an amidino or guanidino group, and these groups are substituted, are so-called prodrug compounds. The unprotected compounds are readily liberated therefrom in the organism by hydrolysis. Preference is given here to prodrug compounds of the formula I in which, for example,

R is CH₂NHCOA, CH₂NHCOOA, CH₂NH—Ar′, C(═NH—OH)—NH₂, C(═NH—O—COA)—NH₂, C(═NH—O—COAr)—NH₂, C(═NH)NH—COOA, C(═NH)NH—COA, C(═NH)NH—COO—Ar, C(═NH)NH—COO—Ar′, NH—C(═NH)NH—COOA, NHC(═NH)NH—COOAr, NHC(═NH)NH—COOAr′,

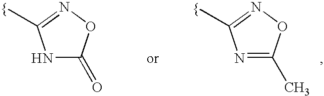

or an amino group which is protected by a conventional amino-protecting group, and the other radicals in the compounds of the formula I have the meanings indicated in claim 1.

R is preferably C(═NH)NH₂, C(═NH—OH)—NH₂ or 5-methyl-1,2,4-oxadiazol-3-yl, N-methoxycarbonylamidino, N-ethoxycarbonylamidino, N-(2,2,2-trichloroethoxycarbonyl)amidino, N-ethylthiocarbonylamidino, N-benzyloxycarbonylamidino, N-phenoxycarbonylamidino, N-(4-fluorophenoxycarbonyl)amidino, N-(4-methoxyphenylthiocarbonyl)amidine, N—[CH₃CO—O—CH(CH₃)—O—CO]-amidine=N-acetoxyethoxycarbonylamidine, N-ethoxycarbonyloxyamidine, N-(N,N-diethylaminoethoxycarbonyl)amidino, N-[(1-methylpiperidin-4-yl)oxycarbonyl]amidino or N-[(pyridin-2-yl)ethoxycarbonyl]amidino, where amidino is particularly preferred. R is particularly preferably in the meta-position of the phenyl ring.

R¹ is preferably, for example, benzyl, methyl, ethyl, propyl, butyl, isopropyl, isobutyl, sec-butyl, pentyl, pent-3-yl, cyclohexylmethyl, 4-fluorobenzyl, ethoxycarbonylmethyl, ethoxycarbonylethyl, (1-methyltetrazol-5-yl)ethyl, methoxyethyl, methoxymethyl or methoxybutyl.

R² is preferably, for example, phenyl which is monosubstituted by SO₂NH₂ or SO₂Me.

A is H or alkyl, where alkyl is unbranched (linear), branched or cyclic and has 1 to 20, preferably 1, 2, 3, 4, 5, 6, 7, 8, 9 or 10 carbon atoms. A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1 -, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. A is very particularly preferably H or alkyl having 1-6 carbon atoms, preferably methyl, ethyl, propyl, isopropyl, butyl, isobutyl, sec-butyl, tert-butyl, pentyl or hexyl.

A is furthermore, for example, cyclopropyl, cyclobutyl, cyclopentyl, cyclohexyl or cycloheptyl.

A′ is alkyl, where alkyl is unbranched (linear), branched or cyclic and has 1 to 10, preferably 1, 2, 3, 4, 5, 6, 7 or 8 carbon atoms. A′ is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A′ is particularly preferably alkyl having 1-6 carbon, atoms, preferably methyl, ethyl, propyl, isopropyl, butyl, isobutyl, sec-butyl, tert-butyl, pentyl or hexyl.

A′ is furthermore, for example, cyclopentyl or cyclohexyl.

A′ is very particularly preferably alkyl having 1-6 carbon atoms, preferably methyl, ethyl, propyl, isopropyl, butyl, isobutyl, sec-butyl, tert-butyl, pentyl or hexyl.

Hal is preferably F, Cl or Br, but also I.

Ar is phenyl or naphthyl which is unsubstituted or monosubstituted, disubstituted or trisubstituted by A′, OH, OA′, NH₂, NHA′, NA′₂, NO₂, CF₃, CN, Hal, NHCOA, COOA, CONH₂, CONHA′, CONA′₂, SA, SOA, SO₂A, SO₂NH₂, SO₂NHA′ or SO₂NA′₂.

Preferred substituents for phenyl or naphthyl are, for example, methyl, ethyl, propyl, butyl, OH, methoxy, ethoxy, propoxy, butoxy, amino, methylamino, dimethylamino, ethylamino, diethylamino, nitro, trifluoromethyl, fluorine, chlorine, acetamido, methoxycarbonyl, ethoxycarbonyl, aminocarbonyl, sulfonamido, methylsulfonamido, ethylsulfonamido, propylsulfonamido, butylsulfonamido, tert-butylsulfonamido, tert-butylaminosulfonyl, dimethylsulfonamido, phenylsulfonamido, carboxyl, dimethylaminocarbonyl, phenylaminocarbonyl, acetyl, propionyl, benzoyl, methylsulfonyl or phenylsulfonyl.

Ar is particularly preferably, for example, unsubstituted phenyl or phenyl which is monosubstituted by SO₂NH₂, SO₂CH₃, fluorine or alkoxy, such as, for example, methoxy.

Ar′ is —(CH₂)ₙ—Ar, where n is preferably 1 or 2, and Ar is as defined above. Very particular preference is given to benzyl which is unsubstituted or monosubstituted, disubstituted or trisubstituted by fluorine and/or chlorine.

Y is preferably, for example, methoxycarbonyl, ethoxycarbonyl or 1 -methyl-tetrazol-5-yl.

In X, n is preferably, for example, 1 or 2.

Het is preferably, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-quinolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated.

Het can thus, for example, also be 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or 4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or 4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, 4-, -5-, -6-, -7- or -8-quinolyl, 1,2,3,4-tetrahydro-1 -, -2-, -3-, 4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxo-methylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

Het is unsubstituted or monosubstituted, disubstituted or trisubstituted, for example, by methyl, methoxy, amino, methylamino, dimethylamino, nitro, cyano, fluorine, chlorine, acetamido, methylsulfonylamino, methoxycarbonyl, aminocarbonyl, acetyl, aminosulfonyl, methylsulfonyl and/or carbonyl oxygen.

Het is particularly preferably, for example, furyl, thienyl, thiazolyl, imidazolyl, 2,1,3-benzothiadiazolyl, oxazolyl, pyridyl, indolyl, 1-methyl-piperidinyl, piperidinyl or pyrrolidinyl, very particularly preferably pyridyl, 1-methylpiperidin-4-yl or piperidin-4-yl.

Accordingly, the invention relates in particular to the compounds of the formula I in which at least one of the said radicals has one of the preferred meanings indicated above. Some preferred groups of compounds may be expressed by the following sub-formulae Ia to Ii, which conform to the formula I and in which the radicals not designated in greater detail have the meaning indicated under the formula I, but in which in Ia R is —C(═NH)—NH₂, which may also be monosubstituted by OH or a conventional amino-protecting group,

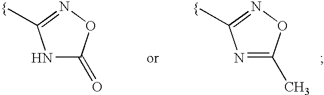

in Ib R is —C(═NH)—NH₂, which may also be monosubstituted by OH or a conventional amino-protecting group,

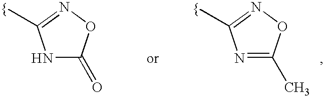

R¹ is unbranched, branched or cyclic alkyl having 1-8 carbon atoms, in which one CH₂ group may be replaced by O, or is Ar, Ar′ or X;

in Ic R is —C(═NH)—NH₂, which may also be monosubstituted by OH or a conventional amino-protecting group,

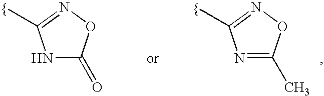

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA;

in Id R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

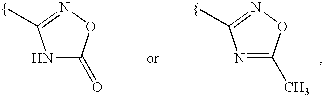

R′ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A;

in Ie R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

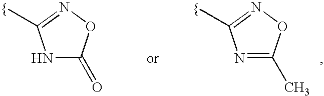

R′ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂;

in If R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

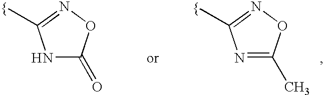

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂ ,

Ar′ is benzyl which is unsubstituted or monosubstituted, disubstituted or trisubstituted by fluorine;

in Ig R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₘN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

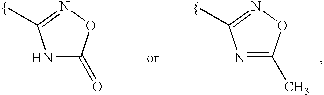

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

A and A′ are each, independently of one another, H or unbranched, branched or cyclic alkyl having 1-8 carbon atoms;

in Ih R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

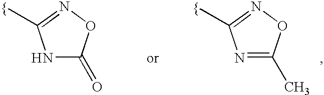

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

Het is a monocyclic saturated or aromatic heterocycle having 1 to 2 N and/or O atoms;

in Ii R is CH₂NH₂, CH₂NHCOA or CH₂NHCOOA, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

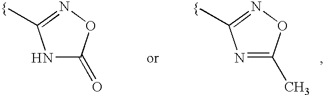

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

Het is a monocyclic saturated or aromatic heterocycle having 1 to 2 N and/or O atoms,

and pharmaceutically tolerated salts and solvates thereof.

The compounds of the formula I and also the starting materials for the preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

Compounds of the formula I can preferably be obtained by liberating compounds of the formula I from one of their functional derivatives by treatment with a solvolysing or hydrogenolysing agent.

Preferred starting materials for the solvolyis or hydrogenolysis are those which conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ is an amino-protecting group, instead of an HN group, and/or those which carry an hydroxyl-protecting group instead of the H atom of an hydroxyl group, for example those which conform to the formula I, but carry a —COOR″ group, in which R″ is an hydroxyl-protecting group, instead of a —COOH group.

Preferred starting materials are also the oxadiazole derivatives which can be converted into the corresponding amidino compounds.

The liberation of the amidino group from its oxadiazole derivative can be carried out, for example, by treatment with hydrogen in the presence of a catalyst (for example water-moist Raney nickel). Suitable solvents are those indicated below, in particular alcohols, such as methanol or ethanol, organic acids, such as acetic acid or propionic acid, or mixtures thereof. The hydrogenolysis is generally carried out at temperatures between about 0 and 100° and pressures between about 1 and 200 bar, preferably at 20-30° (room temperature) and 1-10 bar.

The oxadiazole group is introduced, for example, by reaction of the cyano compounds with hydroxylamine and reaction with phosgene, dialkyl carbonate, chloroformates, N,N′-carbonyldiimidazole or acetic anhydride.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and toluyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols; such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 1.5 and 30° (room temperature).

The BOC, OBut and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethylformamide (DMF); nitrites, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

An SO₂NH₂ group, for example in R², is preferably employed in the form of its tert-butyl derivative. The tert-butyl group is cleaved off, for example, using TFA with or without addition of an inert solvent, preferably with addition of a small amount of anisole (1-10% by volume).

A cyano group is converted into an amidino group by reaction with, for example, hydroxylamine followed by reduction of the N-hydroxyamidine using hydrogen in the presence of a catalyst, such as, for example, Pd/C. In order to prepare an amidine of the formula I (for example Ar=phenyl which is monosubstituted by C(═NH)—NH₂), it is also possible to add ammonia onto a nitrile. The adduction is preferably carried out in a multistep process by, in a manner known per se, a) converting the nitrile into a thioamide using H₂S, converting the thioamide into the corresponding S-alkylimidothioester using an alkylating agent, for example CH₃I, and in turn reacting the thioester with NH₃ to give the amidine, b) converting the nitrile into the corresponding imidoester using an alcohol, for example ethanol, in the presence of HCl, and treating this ester with ammonia, or c) reacting the nitrile with lithium bis(trimethylsilyl)amide, and subsequently hydrolysing the product.

The NH-protected precursors of the compounds of the formula I, in which

R is CN, CH₂NH₂, —NH—C(═NH)—NH₂, —CO—N═C(NH₂)₂, —C(═NH)—NH₂, which is monosubstituted by Ar′, OH, O—COA, O—COAr, OCOOA, OCOO(CH₂)ₙN(A)₂, —COO(CH₂)ₙNA₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group,

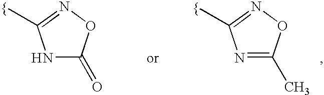

R¹ is as defined in claim 1, and

R² is Br or I,

are prepared, for example, by reaction with the corresponding boronic acid derivatives in a Suzuki reaction. The Suzuki reaction is advantageously carried out with palladium control, preferably by addition of Pd(PPh₃)₄ or Pd(II)Cl₂dppf, in the presence of a base, such as potassium carbonate, in an inert solvent or solvent mixture, for example DMF, at temperatures between 0° and 150°, preferably between 60° and 120°. Depending on the conditions used, the reaction time is between a few minutes and several days. The boronic acid derivatives can be prepared by conventional methods or are commercially available. The reactions can be carried out analogously to the methods indicated in Suzuki et al., J. Am. Chem. Soc. 1989, 111, 314 ff. and in Suzuki et al. Chem. Rev. 1995, 95, 2457 ff.

A base of the formula I can be converted into the associated acid-addition salt using an acid, for example by reaction of equivalent amounts of the base and the acid in an inert solvent, such as ethanol followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate). It is also possible to use physiologically acceptable organic bases, such as, for example, ethanolamine.

Compounds of the formula I according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline) or N-benzenesulfonylproline), or the various optically active camphorsulfonic acids. Also advantage is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Examples of suitable eluents for this purpose are aqueous or alcoholic solvent mixtures, such as, for example, hexane/isopropanol/acetonitrile, for example in the ratio 82:15:3.

The invention furthermore relates to the use of compounds of the formula I and/or their physiologically acceptable salts for the preparation of pharmaceutical preparations, in particular by non-chemical methods. They can be converted here into a suitable dosage form together with at least one solid, liquid and/or semiliquid excipient or assistant and, if desired, in combination with one or more further active ingredients.

The invention thus also relates to pharmaceutical preparations comprising at least one medicament according to one of claims 5 and 6 and, if desired, excipients and/or assistants and, if desired, other active ingredients.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, to prepare injection preparations. The preparations indicated may be sterilised and/or comprise assistants, such as lubricants, preservatives, stabilisers and/or wetting agents, emulsifying agents, salts for modifying the osmotic pressure, buffer substances, colorants and flavours and/or a plurality of further active ingredients, for example one or more vitamins.

The invention also relates to the use of compounds according to claims 1 and 2 and/or their physiologically acceptable salts for the preparation of a medicament for combating thromboembolic illnesses, such as thrombosis, myocardial infarction, arteriosclerosis, inflammation, apoplexia, angina pectoris, restenosis after angioplasty and claudicatio intermittens.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Above and below, all temperatures are given in ° C. In the following examples, ‘conventional work-up’ means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.

Mass spectrometry (MS): EI (electron ionisation) M⁺

FAB (fast atom bombardment) (M+H)⁺

### EXAMPLE 1

Precursors from the n-propyl series

1.1

10.0 ml of triethylamine are added to a solution of 4.6 ml of n-propylamine in 100 ml of THF. 8.5 ml of trifluoroacetic anhydride are subsequently added dropwise. After the mixture has been stirred for 4 hours, it is subjected to conventional work-up, giving 5.58 g of N-propyl-2,2,2-trifluoracetamide (“AA”) as a yellow oil, EI 155.

1.2

13.0 g of caesium carbonate are added to a solution of 5.0 g of “AA” in 200 ml of DMF, and the mixture is stirred at RT for 0.5 hour. 10.0 g of 3-[(3-bromomethyl)phenyl]-5-methyl-1,2,4-oxadiazole (“AB”) are subsequently added dropwise, and the mixture is stirred for a further 18 hours. Conventional work-up gives 9.32 g of 2,2,2-trifluoro-N-propyl-N-[3-[5-methyl-(1,2,4-oxadiazol)-3-yl]benzyl}acetamide (“AC”) as a yellow oil, FAB 328.

1.3

1.9 g of lithium hydroxide and 15 ml of water are added to a solution of 8.5 g of “AC” in 300 ml of methanol, and the mixture is stirred for a further 2.5 hours. Conventional work-up gives 4.51 g of [3-(5-methyl-1,2,4-oxadiazol-3-yl)benzyl]propylamine (“AD”) as a yellow oil, FAB 232.

1.4

1.27 g of 4-bromophenyl isocyanate are added to a solution of 1.48 g of “AD” in 30 ml of acetonitrile, and the mixture is stirred at 70° for 1.5 hours. Conventional work-up gives 2.55 g of 1-[3-(5-methyl-1,2,4-oxadiazol-3-yl)-benzyl]-3-(4-bromophenyl)-1-propylurea (“AE”), m.p. 113-114°, FAB 429/431/433.

### EXAMPLE 2

Precursors from the phenyl series

2.1

Analogously to Example 1.1, 5.0 ml of aniline give 10.25 g of N-phenyl-2,2,2-trifluorocetamide (“BA”), FAB 190.

2.2

Analogously to Example 1.2, 6.0 g of “BA” give 9.37 g of 2,2,2-trifluoro-N-phenyl-N-{3-[5-methyl-(1,2,4-oxadiazol)-3-yl]benzyl}acetamide (“BB”), FAB 362.

2.3

Analogously to Example 1.3, 9.5 g of “BB” give 6.61 g of [3-(5-methyl-1,2,4-oxadiazol-3-yl)benzyl]phenylamine (“BC”), m.p. 75-76°, FAB 266.

2.4

Analogously to Example 1.4, 1.75 g of “BC” give 4.18 g of 1-[3-(5-methyl-1,2,4-oxadiazol-3-yl)benzyl]-3-(4-bromophenyl)-1-phenylurea (“BD”), m.p. 145-146°, FAB 463/465/467.

### EXAMPLE 3

3.1

2.5 g of 2-(tert-butylaminosulfonyl)phenylboronic acid, 10 ml of 2M sodium carbonate solution and 0.12 g of PdCl₂(dppf) are added successively under an N₂ atmosphere to a solution of 0.9 g of “AE” in 50 ml of ethylene glycol dimethyl ether, and the mixture is stirred at 85° for 1.5 hours. Conventional work-up gives 0.53 g of 1-[3-(5-methyl-1,2,4-oxadiazol-3-yl)benzyl]-3-(2′-tert-butylsulfamoylbiphenyl-4-yl)-1-propylurea (“CA”), m.p. 150-151°, EI 561.

3.2

0.5 ml of acetic acid is added to a solution of 0.4 g of “CA” in 5 ml of methanol, 2 g of Raney nickel are added, and the mixture is stirred for 18 hours under a nitrogen atmosphere. After the catalyst has been separated off, conventional work-up gives 0.34 g of 1-(3-amidinobenzyl)-3-(2′-tert-butylsulfamoylbiphenyl4-yl)-1-propylurea (“CB”), m.p. 201-202°, FAB 522.

3.3

A solution of 0.3 g of “CB” in 3 ml of TFA and 0.3 ml of anisole is stirred at RT for 16 hours. Conventional work-up gives 0.27 g of 1-(3-amidino-benzyl)-3-(2′-sulfamoylbiphenyl-4-yl)-1-propylurea, m.p. 166-167°, FAB 466.

Affinity to receptors:

| | | |
|---|---|---|
| IC₅₀ values [nM/litres] | IC₅₀ (factor Xa, human) | = 140.0 |
| | IC₅₀ (TF/VIIa) | = 80.0 |

The following compounds are obtained analogously:

1-(3-amidinobenzyl)-3-(2′-sulfamoylbiphenyl-4-yl)-1-methylurea,

1-(3-amidinobenzyl)-3-(2′-sulfamoylbiphenyl-4-yl)-1-ethylurea,

1-(3-amidinobenzyl)-3-(2′-sulfamoylbiphenyl-4-yl)-1-isopropylurea,

1-(3-amidinobenzyl)-3-(2′-sulfamoylbiphenyl-4-yl)-1-butylurea,

1-(3-amidinobenzyl)-3-(2′-sulfamoylbiphenyl-4-yl)-1-isobutylurea,

1-(3-amidinobenzyl)-3-(2′-sulfamoylbiphenyl-4-yl)-1-pentylurea,

1-(3-amidinobenzyl)-3-(2′-sulfamoylbiphenyl-4-yl)-1-sec-butylurea,

1-(3-amidinobenzyl)-3-(2′-sulfamoylbiphenyl-4-yl)-1-cyclohexylmethylurea,

1-(3-amidinobenzyl)-3-(2′-sulfamoylbiphenyl-4-yl)-1-benzylurea.

### EXAMPLE 4

4.1

Analogously to Example 3.1, 1.4 g of “AE” give 0.98 g of 1-[3-(5-methyl-1,2,4-oxadiazol-3-yl)benzyl]-3-(2′-methylsulfanylbiphenyl-4-yl)-1-propylurea (“DA”), EI 472.

4.2

0.7 g of “DA” and 1.14 g of sodium perborate trihydrate are suspended in 25 ml of acetic acid, and the mixture is stirred at RT for 36 hours. Conventional work-up gives 0.49 g of 1-[3-(5-methyl-1,2,4-oxadiazol-3-yl)benzyl]-3-(2′-methylsulfonylbiphenyl-4-yl)-1-propylurea (“DA”), FAB 505.

4.3

Analogously to Example 3.2, 0.35 g of “DA” gives 0.31 g of 1-(3-amidino-benzyl)-3-(2′-methylsulfonylbiphenyl-4-yl)-1-propylurea, FAB 465.

Affinity to receptors:

| | | |
|---|---|---|
| IC₅₀ values [nM/litres] | IC₅₀ (factor Xa, human) | = 100.0 |
| | IC₅₀ (TF/VIIa) | = 30.0 |

The following compounds are obtained analogously:

1-(3-amidinobenzyl)-3-(2′-methylsulfonylbiphenyl-4-yl)-1-methylurea,

1-(3-amidinobenzyl)-3-(2′-methylsulfonylbiphenyl-4-yl)-1-ethylurea,

1-(3-amidinobenzyl)-3-(2′-methylsulfonylbiphenyl-4-yl)-1-isopropylurea,

1-(3-amidinobenzyl)-3-(2′-methylsulfonylbiphenyl-4-yl)-1-butylurea, FAB 479;

1-(3-amidinobenzyl)-3-(2′-methylsulfonylbiphenyl-4-yl)-1-isobutylurea, FAB 479;

1-(3-amidinobenzyl)-3-(2′-methylsulfonylbiphenyl-4-yl)-1-pentylurea,

1-(3-amidinobenzyl)-3-(2′-methylsulfonylbiphenyl-4-yl)-1-sec-butylurea,

1-(3-amidinobenzyl)-3-(2′-methylsulfonylbiphenyl-4-yl)-1-cyclopentylurea, FAB 491;

1-(3-amidinobenzyl)-3-(2′-methylsulfonylbiphenyl-4-yl)-1-cyclohexylmethylurea,

1-(3-amidinobenzyl)-3-(2′-methylsulfonylbiphenyl-4-yl)-1-benzylurea, FAB 513.

Affinity to receptors:

| | | |
|---|---|---|
| IC₅₀ values [nM/litres] | IC₅₀ (factor Xa, human) | = 80.0 |
| | IC₅₀ (TF/VIIa) | = 26.0 |

### EXAMPLE 5

5.1

Analogously to Example 3.1, 1.0 g of “BD” gives 0.84 g of 1-[3-(5-methyl-1,2,4-oxadiazol-3-yl)benzyl]-3-(2′-tert-butylsulfamoylbiphenyl-4-yl)-1-phenylurea (“EA”), m.p. 191-192°, EI 595.

5.2

Analogously to Example 3.2, 0.5 g of “EA” gives 0.46 g of 1-(3-amidinobenzyl)-3-(2′-tert-butylsulfamoylbiphenyl-4-yl)-1-phenylurea (“EB”), m.p. 140-141°, FAB 556.

5.3

Analogously to Example 3.3, 0.4 g of “EB” gives 0.305 g of 1-(3-amidinobenzyl)-3-(2′-sulfamoylbiphenyl-4-yl)-1-phenylurea, m.p. 201-202°, FAB 500.

Affinity to receptors:

| | | |
|---|---|---|
| IC₅₀ values [nM/litres] | IC₅₀ (factor Xa, human) | = 330.0 |
| | IC₅₀ (TF/VIIa) | = 100.0 |

### EXAMPLE 6

6.1

Analogously to Example 3.1, 1.2 g of “BD” gives 0.82 g of 1-[3-(5-methyl-1,2,4-oxadiazol-3-yl)benzyl]-3-(2′-methylsulfanylbiphenyl-4-yl)-1-phenylurea (“FA”), EI 506.

6.2

Analogously to Example 4.2, 0.65 g of “FA” gives 0.15 g of 1-[3-(5-methyl-1,2,4-oxadiazol-3-yl)benzyl]-3-(2′-methylsulfonylbiphenyl-4-yl)-1-phenylurea (“FB”), FAB 539.

6.3

Analogously to Example 3.2, 0.12 g of “FB” gives 0.11 g of 1-(3-amidinobenzyl)-3-(2′-methylsulfonylbiphenyl-4-yl)-1-phenylurea, FAB 499.

Affinity to receptors:

| | | |
|---|---|---|
| IC₅₀ values [nM/litres] | IC₅₀ (factor Xa, human) | = 300.0 |
| | IC₅₀ (TF/VIIa) | = 70.0 |

### EXAMPLE 7

The reactions described in this example are carried out analogously to the working procedure of S. M. Rahmathullah et al. in J. Med. Chem. 1999, 42, 3994-4000. The corresponding acid chlorides are firstly derivatised to the 4-nitrophenyl carbonate compounds, which are then reacted further with the amidino compounds.

Starting from methyl chloroformate and reaction of the following “amidino compounds”:

1-(3-amidinobenzyl)-3-(2′-sulfamoylbiphenyl-4-yl)-1-propylurea,

1-(3-amidinobenzyl)-3-(2′-sulfamoylbiphenyl-4-yl)-1-phenylurea,

1-(3-amidinobenzyl)-3-(2′-sulfamoylbiphenyl-4-yl)-1-methylurea,

1-(3-amidinobenzyl)-3-(2′-sulfamoylbiphenyl-4-yl)-1-ethylurea,

1-(3-amidinobenzyl)-3-(2′-sulfamoylbiphenyl-4-yl)-1-isopropylurea,

1-(3-amidinobenzyl)-3-(2′-sulfamoylbiphenyl-4-yl)-1-butylurea,

1-(3-amidinobenzyl)-3-(2′-sulfamoylbiphenyl-4-yl)-1-isobutylurea,

1-(3-amidinobenzyl)-3-(2′-sulfamoylbiphenyl-4-yl)-1-pentylurea,

1-(3-amidinobenzyl)-3-(2′-sulfamoylbiphenyl-4-yl)-1-sec-butylurea,

1-(3-amidinobenzyl)-3-(2′-sulfamoylbiphenyl-4-yl)-1-cyclohexylmethylurea,

1-(3-amidinobenzyl)-3-(2′-sulfamoylbiphenyl-4-yl)-1-cyclopentylurea,

1-(3-amidinobenzyl)-3-(2′-sulfamoylbiphenyl-4-yl)-1-benzylurea

gives

1-(3-N-methoxycarbonylamidinobenzyl)-3-(2′-sulfamoylbiphenyl-4-yl)-1-propylurea,

1-(3-N-methoxycarbonylamidinobenzyl)-3-(2′-sulfamoylbiphenyl-4-yl)-1-phenylurea,

1-(3-N-methoxycarbonylamidinobenzyl)-3-(2′-sulfamoylbiphenyl-4-yl)-1-methylurea,

1-(3-N-methoxycarbonylamidinobenzyl)-3-(2′-sulfamoylbiphenyl-4-yl)-1-ethylurea,

1-(3-N-methoxycarbonylamidinobenzyl)-3-(2′-sulfamoylbiphenyl-4-yl)-1-isopropylurea,

1-(3-N-methoxycarbonylamidinobenzyl)-3-(2′-sulfamoylbiphenyl-4-yl)-1-butylurea,

1-(3-N-methoxycarbonylamidinobenzyl)-3-(2′-sulfamoylbiphenyl-4-yl)-1-isobutylurea,

1-(3-N-methoxycarbonylamidinobenzyl)-3-(2′-sulfamoylbiphenyl-4-yl)-1-pentylurea,

1-(3-N-methoxycarbonylamidinobenzyl)-3-(2′-sulfamoylbiphenyl-4-yl)-1-sec-butylurea,

1-(3-N-methoxycarbonylamidinobenzyl)-3-(2′-sulfamoylbiphenyl-4-yl)-1-cyclohexylmethylurea,

1-(3-N-methoxycarbonylamidinobenzyl)-3-(2′-sulfamoylbiphenyl-4-yl)-1-cyclopentylurea,

1-(3-N-methoxycarbonylamidinobenzyl)-3-(2′-sulfamoylbiphenyl-4-yl)-1-benzylurea.

Starting from thioethyl chloroformate and reaction of the “amidino compounds” gives

1-(3-N-ethylthiocarbonylamidinobenzyl)-3-(2′-sulfamoylbiphenyl-4-yl)-1-propylurea,

1-(3-N-ethylthiocarbonylamidinobenzyl)-3-(2′-sulfamoylbiphenyl-4-yl)-1-phenylurea,

1-(3-N-ethylthiocarbonylamidinobenzyl)-3-(2′-sulfamoylbiphenyl-4-yl)-1-methylurea,

1-(3-N-ethylthiocarbonylamidinobenzyl)-3-(2′-sulfamoylbiphenyl-4-yl)-1-ethylurea,

1-(3-N-ethylthiocarbonylamidinobenzyl)-3-(2′-sulfamoylbiphenyl-4-yl)-1-isopropylurea,

1-(3-N-ethylthiocarbonylamidinobenzyl)-3-(2′-sulfamoylbiphenyl-4-yl)-1-butylurea,

1-(3-N-ethylthiocarbonylamidinobenzyl)-3-(2′-sulfamoylbiphenyl-4-yl)-1-isobutylurea,

1-(3-N-ethylthiocarbonylamidinobenzyl)-3-(2′-sulfamoylbiphenyl-4-yl)-1-pentylurea,

1-(3-N-ethylthiocarbonylamidinobenzyl)-3-(2′-sulfamoylbiphenyl-4-yl)-1-sec-butylurea,

1-(3-N-ethylthiocarbonylamidinobenzyl)-3-(2′-sulfamoylbiphenyl-4-yl)-1-cyclohexylmethylurea,

1-(3-N-ethylthiocarbonylamidinobenzyl)-3-(2′-sulfamoylbiphenyl-4-yl)-1-cyclopentylurea,

1-(3-N-ethylthiocarbonylamidinobenzyl)-3-(2′-sulfamoylbiphenyl-4-yl)-1-benzylurea.

Starting from 2,2,2-trichloroethyl chloroformate and reaction of the “amidino compounds” gives

1-(3-N-(2,2,2-trichloroethoxycarbonyl)amidinobenzyl)-3-(2′-sulfamoyl-biphenyl-4-yl)-1-propylurea,

1-(3-N-(2,2,2-trichloroethoxycarbonyl)amidinobenzyl)-3-(2′-sulfamoylbiphenyl-4-yl)-1-phenylurea,

1-(3-N-(2,2,2-trichloroethoxycarbonyl)amidinobenzyl)-3-(2′-sulfamoylbiphenyl-4-yl)-1-methylurea,

1-(3-N-(2,2,2-trichloroethoxycarbonyl)amidinobenzyl)-3-(2′-sulfamoylbiphenyl-4-yl)-1-ethylurea,

1-(3-N-(2,2,2-trichloroethoxycarbonyl)amidinobenzyl)-3-(2′-sulfamoylbiphenyl-4-yl)-1-isopropylurea,

1-(3-N-(2,2,2-trichloroethoxycarbonyl)amidinobenzyl)-3-(2′-sulfamoylbiphenyl-4-yl)-1-butylurea,

1-(3-N-(2,2,2-trichloroethoxycarbonyl)amidinobenzyl)-3-(2′-sulfamoylbiphenyl-4-yl)-1-isobutylurea,

1-(3-N-(2,2,2-trichloroethoxycarbonyl)amidinobenzyl)-3-(2′-sulfamoylbiphenyl-4-yl)-1-pentylurea,

1-(3-N-(2,2,2-trichloroethoxycarbonyl)amidinobenzyl)-3-(2′-sulfamoylbiphenyl-4-yl)-1-sec-butylurea,

1-(3-N-(2,2,2-trichloroethoxycarbonyl)amidinobenzyl)-3-(2′-sulfamoylbiphenyl-4-yl)-1-cyclohexylmethylurea,

1-(3-N-(2,2,2-trichloroethoxycarbonyl)amidinobenzyl)-3-(2′-sulfamoylbiphenyl-4-yl)-1-cyclopentylurea,

1-(3-N-(2,2,2-trichloroethoxycarbonyl)amidinobenzyl)-3-(2′-sulfamoylbiphenyl-4-yl)-1-benzylurea.

Starting from benzyl chloroformate and reaction of the “amidino compounds” gives

1-(3-N-benzyloxycarbonylamidinobenzyl)-3-(2′-sulfamoylbiphenyl-4-yl)-1-propylurea,

1-(3-N-benzyloxycarbonylamidinobenzyl)-3-(2′-sulfamoylbiphenyl-4-yl)-1-phenylurea,

1-(3-N-benzyloxycarbonylamidinobenzyl)-3-(2′-sulfamoylbiphenyl-4-yl)-1-methylurea,

1-(3-N-benzyloxycarbonylamidinobenzyl)-3-(2′-sulfamoylbiphenyl-4-yl)-1-ethylurea,

1-(3-N-benzyloxycarbonylamidinobenzyl)-3-(2′-sulfamoylbiphenyl-4-yl)-1-isopropylurea,

1-(3-N-benzyloxycarbonylamidinobenzyl)-3-(2′-sulfamoylbiphenyl-4-yl)-1-butylurea,

1-(3-N-benzyloxycarbonylamidinobenzyl)-3-(2′-sulfamoylbiphenyl-4-yl)-1-isobutylurea,

1-(3-N-benzyloxycarbonylamidinobenzyl)-3-(2′-sulfamoylbiphenyl-4-yl)-1-pentylurea,

1-(3-N-benzyloxycarbonylamidinobenzyl)-3-(2′-sulfamoylbiphenyl-4-yl)-1-sec-butylurea,

1-(3-N-benzyloxycarbonylamidinobenzyl)-3-(2′-sulfamoylbiphenyl-4-yl)-1-cyclohexylmethylurea,

1-(3-N-benzyloxycarbonylamidinobenzyl)-3-(2′-sulfamoylbiphenyl-4-yl)-1-cyclopentylurea,

1-(3-N-benzyloxycarbonylamidinobenzyl)-3-(2′-sulfamoylbiphenyl-4-yl)-1-benzylurea.

Starting from phenyl chloroformate and the reaction of the “amidino compounds” gives

1-(3-N-phenoxycarbonylamidinobenzyl)-3-(2′-sulfamoylbiphenyl-4-yl)-1-propylurea,

1-(3-N-phenoxycarbonylamidinobenzyl)-3-(2′-sulfamoylbiphenyl-4-yl)-1-phenylurea,

1-(3-N-phenoxycarbonylamidinobenzyl)-3-(2′-sulfamoylbiphenyl-4-yl)-1-methylurea,

1-(3-N-phenoxycarbonylamidinobenzyl)-3-(2′-sulfamoylbiphenyl-4-yl)-1-ethylurea,

1-(3-N-phenoxycarbonylamidinobenzyl)-3-(2′-sulfamoylbiphenyl-4-yl)-1-isopropylurea,

1-(3-N-phenoxycarbonylamidinobenzyl)-3-(2′-sulfamoylbiphenyl-4-yl)-1-butylurea,

1-(3-N-phenoxycarbonylamidinobenzyl)-3-(2′-sulfamoylbiphenyl-4-yl)-1-isobutylurea,

1-(3-N-phenoxycarbonylamidinobenzyl)-3-(2′-sulfamoylbiphenyl-4-yl)-1-pentylurea,

1-(3-N-phenoxycarbonylamidinobenzyl)-3-(2′-sulfamoylbiphenyl-4-yl)-1-sec-butylurea,

1-(3-N-phenoxycarbonylamidinobenzyl)-3-(2′-sulfamoylbiphenyl-4-yl)-1-cyclohexylmethylurea,

1-(3-N-phenoxycarbonylamidinobenzyl)-3-(2′-sulfamoylbiphenyl-4-yl)-1-cyclopentylurea,

1-(3-N-phenoxycarbonylamidinobenzyl)-3-(2′-sulfamoylbiphenyl-4-yl)-1-benzylurea.

Starting from 4-fluorophenyl chloroformate and reaction of the “amidino compounds” gives

1-(3-N-(4-fluorophenoxycarbonyl)amidinobenzyl)-3-(2′-sulfamoylbiphenyl-4-yl)-1-propylurea,

1-(3-N-(4-fluorophenoxycarbonyl)amidinobenzyl)-3-(2′-sulfamoylbiphenyl-4-yl)-1-phenylurea,

1-(3-N-(4-fluorophenoxycarbonyl)amidinobenzyl)-3-(2′-sulfamoylbiphenyl-4-yl)-1-methylurea,

1-(3-N-(4-fluorophenoxycarbonyl)amidinobenzyl)-3-(2′-sulfamoylbiphenyl-4-yl)-1-ethylurea,

1-(3-N-(4-fluorophenoxycarbonyl)amidinobenzyl)-3-(2′-sulfamoylbiphenyl-4-yl)-1-isopropylurea,

1-(3-N-(4-fluorophenoxycarbonyl)amidinobenzyl)-3-(2′-sulfamoylbiphenyl-4-yl)-1-butylurea,

1-(3-N-(4-fluorophenoxycarbonyl)amidinobenzyl)-3-(2′-sulfamoylbiphenyl-4-yl)-1-isobutylurea,

1-(3-N-(4-fluorophenoxycarbonyl)amidinobenzyl)-3-(2′-sulfamoylbiphenyl-4-yl)-1-pentylurea,

1-(3-N-(4-fluorophenoxycarbonyl)amidinobenzyl)-3-(2′-sulfamoylbiphenyl-4-yl)-1-sec-butylurea,

1-(3-N-(4-fluorophenoxycarbonyl)amidinobenzyl)-3-(2′-sulfamoylbiphenyl-4-yl)-1-cyclohexylmethylurea,

1-(3-N-(4-fluorophenoxycarbonyl)amidinobenzyl)-3-(2′-sulfamoylbiphenyl-4-yl)-1-cyclopentylurea,

1-(3-N-(4-fluorophenoxycarbonyl)amidinobenzyl)-3-(2′-sulfamoylbiphenyl-4-yl)-1-benzylurea.

Starting from thio-4-methoxyphenyl chloroformate and the reaction of the “amidino compounds” gives

1-(3-N-(4-methoxyphenylthiocarbonyl)amidinobenzyl)-3-(2′-sulfamoylbiphenyl-4-yl)-1-propylurea,

1-(3-N-(4-methoxyphenylthiocarbonyl)amidinobenzyl)-3-(2′-sulfamoylbiphenyl-4-yl)-1-phenylurea,

1-(3-N-(4-methoxyphenylthiocarbonyl)amidinobenzyl)-3-(2′-sulfamoylbiphenyl-4-yl)-1-methylurea,

1-(3-N-(4-methoxyphenylthiocarbonyl)amidinobenzyl)-3-(2′-sulfamoylbiphenyl-4-yl)-1-ethylurea,

1-(3-N-(4-methoxyphenylthiocarbonyl)amidinobenzyl)-3-(2′-sulfamoylbiphenyl-4-yl)-1-isopropylurea,

1-(3-N-(4-methoxyphenylthiocarbonyl)amidinobenzyl)-3-(2′-sulfamoylbiphenyl-4-yl)-1-butylurea,

1-(3-N-(4-methoxyphenylthiocarbonyl)amidinobenzyl)-3-(2′-sulfamoylbiphenyl-4-yl)-1-isobutylurea,

1-(3-N-(4-methoxyphenylthiocarbonyl)amidinobenzyl)-3-(2′-sulfamoylbiphenyl-4-yl)-1-pentylurea,

1-(3-N-(4-methoxyphenylthiocarbonyl)amidinobenzyl)-3-(2′-sulfamoylbiphenyl-4-yl)-1-sec-butylurea,

1-(3-N-(4-methoxyphenylthiocarbonyl)amidinobenzyl)-3-(2′-sulfamoyl-biphenyl-4-yl )-1-cyclohexylmethylurea,

1-(3-N-(4-methoxyphenylthiocarbonyl)amidinobenzyl)-3-(2′-sulfamoylbiphenyl-4-yl)-1-cyclopentylurea,

1-(3-N-(4-methoxyphenylthiocarbonyl)amidinobenzyl)-3-(2′-sulfamoylbiphenyl-4-yl)-1-benzylurea.

Reaction of the “amidino compounds” with 1-acetoxyethyl 4-nitrophenyl-carbonate gives

1-(3-N-acetoxyethoxycarbonylamidinobenzyl)-3-(2′-sulfamoylbiphenyl-4-yl)-1-propylurea,

1-(3-N-acetoxyethoxycarbonylamidinobenzyl)-3-(2′-sulfamoylbiphenyl-4-yl)-1-phenylurea,

1-(3-N-acetoxyethoxycarbonylamidinobenzyl)-3-(2′-sulfamoylbiphenyl-4-yl)-1-methylurea,

1-(3-N-acetoxyethoxycarbonylamidinobenzyl)-3-(2′-sulfamoylbiphenyl-4-yl)-1-ethylurea,

1-(3-N-acetoxyethoxycarbonylamidinobenzyl)-3-(2′-sulfamoylbiphenyl-4-yl)-1-isopropylurea,

1-(3-N-acetoxyethoxycarbonylamidinobenzyl)-3-(2′-sulfamoylbiphenyl-4-yl)-1-butylurea,

1-(3-N-acetoxyethoxycarbonylamidinobenzyl)-3-(2′-sulfamoylbiphenyl-4-yl)-1-isobutylurea,

1-(3-N-acetoxyethoxycarbonylamidinobenzyl)-3-(2′-sulfamoylbiphenyl-4-yl)-1-pentylurea,

1-(3-N-acetoxyethoxycarbonylamidinobenzyl)-3-(2′-sulfamoylbiphenyl-4-yl)-1-sec-butylurea,

1-(3-N-acetoxyethoxycarbonylamidinobenzyl)-3-(2′-sulfamoylbiphenyl-4-yl)-1-cyclohexylmethylurea,

1-(3-N-acetoxyethoxycarbonylamidinobenzyl)-3-(2′-sulfamoylbiphenyl-4-yl)-1-cyclopentylurea,

1-(3-N-acetoxyethoxycarbonylamidinobenzyl)-3-(2′-sulfamoylbiphenyl-4-yl)-1-benzylurea.

### EXAMPLE 8

The reaction is carried out analogously to S. M. Rahmathullah et al. in J. Med. Chem. 1999, 42, 3994-4000.

Reaction of ethyl chloroformate and the following “N-hydroxyamidino compounds”:

1-(3-N-hydroxyamidinobenzyl)-3-(2′-sulfamoylbiphenyl-4-yl)-1-propylurea,

1-(3-N-hydroxyamidinobenzyl)-3-(2′-sulfamoylbiphenyl-4-yl)-1-phenylurea,

1-(3-N-hydroxyamidinobenzyl)-3-(2′-sulfamoylbiphenyl-4-yl)-1-methylurea,

1-(3-N-hydroxyamidinobenzyl)-3-(2′-sulfamoylbiphenyl-4-yl)-1-ethylurea,

1-(3-N-hydroxyamidinobenzyl)-3-(2′-sulfamoylbiphenyl-4-yl)-1-isopropylurea,

1-(3-N-hydroxyamidinobenzyl)-3-(2′-sulfamoylbiphenyl-4-yl)-1-butylurea,

1-(3-N-hydroxyamidinobenzyl)-3-(2′-sulfamoylbiphenyl-4-yl)-1-isobutylurea,

1-(3-N-hydroxyamidinobenzyl)-3-(2′-sulfamoylbiphenyl-4-yl)-1-pentylurea,

1-(3-N-hydroxyamidinobenzyl)-3-(2′-sulfamoylbiphenyl-4-yl)-1-sec-butylurea,

1-(3-N-hydroxyamidinobenzyl)-3-(2′-sulfamoylbiphenyl-4-yl)-1-cyclohexylmethylurea,

1-(3-N-hydroxyamidinobenzyl)-3-(2′-sulfamoylbiphenyl-4-yl)-1-cyclopentylurea,

1-(3-N-hydroxyamidinobenzyl)-3-(2′-sulfamoylbiphenyl-4-yl)-1-benzylurea

gives

1-(3-N-ethoxycarbonyloxyamidinobenzyl)-3-(2′-sulfamoylbiphenyl-4-yl)-1-propylurea,

1-(3-N-ethoxycarbonyloxyamidinobenzyl)-3-(2′-sulfamoylbiphenyl-4-yl)-1-phenylurea,

1-(3-N-ethoxycarbonyloxyamidinobenzyl)-3-(2′-sulfamoylbiphenyl-4-yl)-1-methylurea,

1-(3-N-ethoxycarbonyloxyamidinobenzyl)-3-(2′-sulfamoylbiphenyl-4-yl)-1-ethylurea,

1-(3-N-ethoxycarbonyloxyamidinobenzyl)-3-(2′-sulfamoylbiphenyl-4-yl)-1-isopropylurea,

1-(3-N-ethoxycarbonyloxyamidinobenzyl)-3-(2′-sulfamoylbiphenyl-4-yl)-1-butylurea,

1-(3-N-ethoxycarbonyloxyamidinobenzyl)-3-(2′-sulfamoylbiphenyl-4-yl)-1-isobutylurea,

1-(3-N-ethoxycarbonyloxyamidinobenzyl)-3-(2′-sulfamoylbiphenyl-4-yl)-1-pentylurea,

1-(3-N-ethoxycarbonyloxyamidinobenzyl)-3-(2′-sulfamoylbiphenyl-4-yl)-1-sec-butylurea,

1-(3-N-ethoxycarbonyloxyamidinobenzyl)-3-(2′-sulfamoylbiphenyl-4-yl)-1-cyclohexylmethylurea,

1-(3-N-ethoxycarbonyloxyamidinobenzyl)-3-(2′-sulfamoylbiphenyl-4-yl)-1-cyclopentylurea,

1-(3-N-ethoxycarbonyloxyamidinobenzyl)-3-(2′-sulfamoylbiphenyl-4-yl)-1-benzylurea.

### EXAMPLE 9

The following compounds are obtained analogously to Example 7:

1-(3-N-(N,N-diethylaminoethoxycarbonyl)amidinobenzyl)-3-(2′-sulfamoylbiphenyl-4-yl)-1-propylurea,

1-(3-N-(N,N-diethylaminoethoxycarbonyl)amidinobenzyl)-3-(2′-sulfamoylbiphenyl-4-yl)-1-phenylurea,

1-(3-N-(N,N-diethylaminoethoxycarbonyl)amidinobenzyl)-3-(2′-sulfamoylbiphenyl-4-yl)-1-methylurea,

1-(3-N-(N,N-diethylaminoethoxycarbonyl)amidinobenzyl)-3-(2′-sulfamoylbiphenyl-4-yl)-1-ethylurea,

1-(3-N-(N,N-diethylaminoethoxycarbonyl)amidinobenzyl)-3-(2′-sulfamoyl-biphenyl-4-yl)-1-isopropylurea,

1-(3-N-(N,N-diethylaminoethoxycarbonyl)amidinobenzyl)-3-(2′-sulfamoylbiphenyl-4-yl)-1-butylurea,

1-(3-N-(N,N-diethylaminoethoxycarbonyl)amidinobenzyl)-3-(2′-sulfamoylbiphenyl-4-yl)-1-isobutylurea,

1-(3-N-(N,N-diethylaminoethoxycarbonyl)amidinobenzyl)-3-(2′-sulfamoylbiphenyl-4-yl)-1-pentylurea,

1-(3-N-(N,N-diethylaminoethoxycarbonyl)amidinobenzyl)-3-(2′-sulfamoylbiphenyl-4-yl)-1-sec-butylurea,

1-(3-N-(N,N-diethylaminoethoxycarbonyl)amidinobenzyl)-3-(2′-sulfamoylbiphenyl-4-yl)-1-cyclohexylmethylurea,

1-(3-N-(N,N-diethylaminoethoxycarbonyl)amidinobenzyl)-3-(2′-sulfamoylbiphenyl-4-yl)-1-cyclopentylurea,

1-(3-N-(N,N-diethylaminoethoxycarbonyl)amidinobenzyl)-3-(2′-sulfamoylbiphenyl-4-yl)-1-benzylurea,

1-(3-N-(N-methylpiperidin-4-yloxycarbonyl)amidinobenzyl)-3-(2′-sulfamoylbiphenyl-4-yl)-1-propylurea,

1-(3-N-(N-methylpiperidin-4-yloxycarbonyl)amidinobenzyl)-3-(2′-sulfamoylbiphenyl-4-yl)-1-phenylurea,

1-(3-N-(N-methylpiperidin-4-yloxycarbonyl)amidinobenzyl)-3-(2′-sulfamoyl-biphenyl-4-yl)-1-methylurea,

1-(3-N-(N-methylpiperidin-4-yloxycarbonyl)amidinobenzyl)-3-(2′-sulfamoylbiphenyl-4-yl)-1-ethylurea,

1-(3-N-(N-methylpiperidin-4-yloxycarbonyl)amidinobenzyl)-3-(2′-sulfamoylbiphenyl-4-yl)-1-isopropylurea,

1-(3-N-(N-methylpiperidin-4-yloxycarbonyl)amidinobenzyl)-3-(2′-sulfamoylbiphenyl-4-yl)-1-butylurea,

1-(3-N-(N-methylpiperidin-4-yloxycarbonyl)amidinobenzyl)-3-(2′-sulfamoylbiphenyl-4-yl)-1-isobutylurea,

1-(3-N-(N-methylpiperidin-4-yloxycarbonyl)amidinobenzyl)-3-(2′-sulfamoylbiphenyl-4-yl)-1-pentylurea,

1-(3-N-(N-methylpiperidin-4-yloxycarbonyl)amidinobenzyl)-3-(2′-sulfamoylbiphenyl-4-yl)-1-sec-butylurea,

1-(3-N-(N-methylpiperidin-4-yloxycarbonyl)amidinobenzyl)-3-(2′-sulfamoylbiphenyl-4-yl)-1-cyclohexylmethylurea,

1-(3-N-(N-methylpiperidin-4-yloxycarbonyl)amidinobenzyl)-3-(2′-sulfamoylbiphenyl-4-yl)-1-cyclopentylurea,

1-(3-N-(N-methylpiperidin-4-yloxycarbonyl)amidinobenzyl)-3-(2′-sulfamoylbiphenyl-4-yl)-1-benzylurea,

1-(3-N-(pyridin-2-yl-ethoxycarbonyl)amidinobenzyl)-3-(2′-sulfamoylbiphenyl-4-yl)-1-propylurea,

1-(3-N-(pyridin-2-yl-ethoxycarbonyl)amidinobenzyl)-3-(2′-sulfamoylbiphenyl-4-yl)-1-phenylurea,

1-(3-N-(pyridin-2-yl-ethoxycarbonyl)amidinobenzyl)-3-(2′-sulfamoylbiphenyl-4-yl)-1-methylurea,

1-(3-N-(pyridin-2-yl-ethoxycarbonyl)amidinobenzyl)-3-(2′-sulfamoylbiphenyl-4-yl)-1-ethylurea,

1-(3-N-(pyridin-2-yl-ethoxycarbonyl)amidinobenzyl)-3-(2′-sulfamoylbiphenyl-4-yl)-1-isopropylurea,

1-(3-N-(pyridin-2-yl-ethoxycarbonyl)amidinobenzyl)-3-(2′-sulfamoylbiphenyl-4-yl)-1-butylurea,

1-(3-N-(pyridin-2-yl-ethoxycarbonyl)amidinobenzyl)-3-(2′-sulfamoyl-biphenyl-4-yl)-1-isobutylurea,

1-(3-N-(pyridin-2-yl-ethoxycarbonyl)amidinobenzyl)-3-(2′-sulfamoylbiphenyl-4-yl)-1-pentylurea,

1-(3-N-(pyridin-2-yl-ethoxycarbonyl)amidinobenzyl)-3-(2′-sulfamoylbiphenyl-4-yl)-1-sec-butylurea,

1-(3-N-(pyridin-2-yl-ethoxycarbonyl)amidinobenzyl)-3-(2′-sulfamoylbiphenyl-4-yl)-1-cyclohexylmethylurea,

1-(3-N-(pyridin-2-yl-ethoxycarbonyl)amidinobenzyl)-3-(2′-sulfamoylbiphenyl-4-yl)-1-cyclopentylurea,

1-(3-N-(pyridin-2-yl-ethoxycarbonyl)amidinobenzyl)-3-(2′-sulfamoylbiphenyl-4-yl)-1-benzylurea.

### EXAMPLE 10

Reaction of 2,2,2-trifluoroacetamide with ethyl bromoacetate analogously to 1.1 and further reaction analogously to 1.2, 1.3, 1.4, 3.1, 3.2 and 3.3 gives 1-(3-amidinobenzyl)-3-(2′-sulfamoylbiphenyl-4-yl)-1-ethoxycarbonylmethylurea.

Analogous reaction with methyl bromopropionate gives the compound 1-(3-amidinobenzyl)-3-(2′-sulfamoylbiphenyl-4-yl)-1-methoxycarbonylethylurea, FAB 509.

### EXAMPLE 11

Preparation of 1-(3-amidinobenzyl)-3-(2′-sulfamoylbiphenyl-4-yl)-1-(1-methyltetrazol-5-ylethyl)urea (“GA”):

Analogously to the above examples, use of 3-bromopropionitrile gives the compound 1-(3-(5-methyl-1,2,4-oxadiazol-3-yl)benzyl)-3-(2′-sulfamoylbiphenyl-4-yl)-1-(2-cyanoethyl)urea.

Conversion of the cyano group into the 1H-tetrazol-5-yl group is carried out by conventional methods by reaction with sodium azide or trimethylsilyl azide, giving 1-(3-(5-methyl-1,2,4-oxadiazol-3-yl)benzyl)-3-(2′-sulfamoylbiphenyl-4-yl)-1-(2-(1H-tetrazol-5-yl)ethyl)urea.

Methylation using methyl iodide followed by hydrogenation in methanol/acetic acid with Raney nickel catalysis gives, after removal of the catalyst and conventional work-up, the compound “GA”.

Starting from

2-methoxyethyl bromide,

1-bromodimethyl ether and

4-methoxybutyl bromide,

the following compounds are obtained analogously:

1-(3-amidinobenzyl)-3-(2′-sulfamoylbiphenyl-4-yl)-1-methoxyethylurea,

1-(3-amidinobenzyl)-3-(2′-sulfamoylbiphenyl-4-yl)-1-methoxymethylurea,

1-(3-amidinobenzyl)-3-(2′-sulfamoylbiphenyl-4-yl)-1-methoxybutylurea.

The examples below relate to pharmaceutical preparations:

### EXAMPLE A

Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C

Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

Ointment

500 mg of an active ingredient of the formula I are mixed with 99.5 g of Vaseline under aseptic conditions.

### EXAMPLE E

Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

Coated-Tablets

Tablets are pressed analogously to Example E and subsequently coated in a conventional manner with a coating of sucrose, potato starch, talc, tragacanth and dye.

### EXAMPLE G

Capsules

2 kg of active ingredient of the formula I are introduced into hard gelatine capsules in a conventional manner in such a way that each capsule contains 20 mg of the active ingredient.

### EXAMPLE H

Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 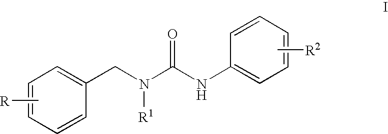
in which
R is CN, CH₂NH₂, —NH—C(═NH)—NH₂, —CO—N═C(NH₂)₂, —C(═NH)—NH₂, which may also be monosubstituted by Ar′, OH, O—COA, O—COAr, OCOOA, OCOO(CH₂)ₙN(A)₂, —COO(CH₂)ₙNA₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group 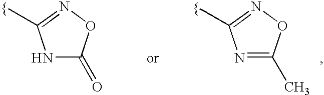
R¹ is R⁴ Ar, Ar′ or X,
R² is phenyl which is monosubstituted by SA, SOA, SO₂A, SONHA, SO₂NHA, CF₃, COOA, CH₂NHA, CN or OA,
R³ is CHal₃, OCOA or 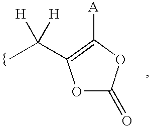
R⁴ is alkyl having 1-20 carbon atoms in which one or two CH₂ groups may be replaced by O or S atoms and/or by —CH═CH— groups and/or 1-7 H atoms may be replaced by F,
A is H or alkyl having 1-20 carbon atoms,
A′ is alkyl having 1-10 carbon atoms,
Ar is phenyl or naphthyl, each of which is unsubstituted or monosubstituted, disubstituted or trisubstituted by A′, OH, OA′, NH₂, NHA′, NA′₂, NO₂, CF₃, CN, Hal, NHCOA, COOA, CONH₂, CONHA′, CONA′₂, SA, SOA, SO₂A, SO₂NH₂, SO₂NHA′ or SO₂NA′₂,
Ar′ is (CH₂)ₙ—Ar,
Het is a monocyclic or bicyclic saturated, unsaturated or aromatic heterocycle having 1 to 4 N, O and/or S atoms, which may be unsubstituted or monosubstituted, disubstituted or trisubstituted by A′, OA′, NH₂, NHA′, NA′₂, NO₂, CN, Hal, NHCOA′, NHSO₂A′, COOA, CONH₂, CONHA′, CONA′₂, COA, SO₂NH₂, SA′, SOA′, SO₂A′ and/or carbonyl oxygen,
X is (CH₂)ₙY,
Y is COOA or 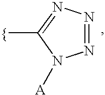
Hal is F, Cl, Br or I,
n is 1, 2, 3, 4, 5 or 6, and
m is 0 or 1,
and their pharmaceutically tolerated salts and solvates.

2. Compounds according to claim 1, in which
R is —C(═NH)—NH₂, which may also be monosubstituted by OH or a conventional amino-protecting group, 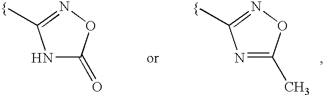
and their pharmaceutically tolerated salts, solvates and stereoisomers.

3. Compounds according to claim 1, in which
R is —C(═NH)—NH₂, which may also be monosubstituted by OH or a conventional amino-protecting group, 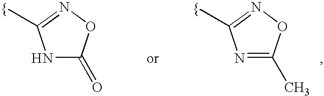
R¹ is unbranched, branched or cyclic alkyl having 1-8 carbon atoms, in which one CH₂ group may be replaced by O, or is Ar, Ar′ or X,
and their pharmaceutically tolerated salts, solvates and stereoisomers.

4. Compounds according to claim 1, in which
R is —C(═NH)—NH₂, which may also be monosubstituted by OH or a conventional amino-protecting group, 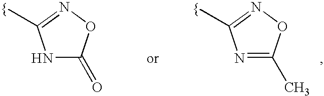
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
and their pharmaceutically tolerated salts, solvates and stereoisomers.

5. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 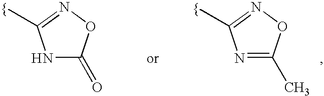
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
and their pharmaceutically tolerated salts, solvates and stereoisomers.

6. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 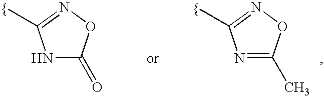
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
and their pharmaceutically tolerated salts, solvates and stereoisomers.

7. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 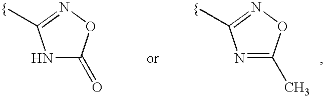
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
and their pharmaceutically tolerated salts, solvates and stereoisomers.

8. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 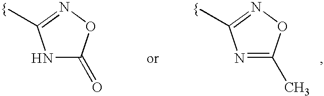
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
A and A′ are each, independently of one another, H or unbranched, branched or cyclic alkyl having 1-8 carbon atoms;
and their pharmaceutically tolerated salts, solvates and stereoisomers.

9. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 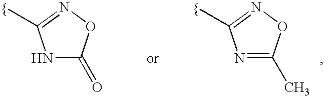
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
Het is a monocyclic saturated or aromatic heterocycle having 1 to 2 N and/or O atoms,
and their pharmaceutically tolerated salts, solvates and stereoisomers.

10. Compounds according to claim 1, in which
R is CH₂NH₂, CH₂NHCOA or CH₂NHCOOA, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂-R³, COOA, COSA, COSAr, COOAr, COOAr′, COA, COAr, COAr′ or by a conventional amino-protecting group, 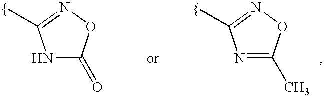
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
Het is a monocyclic saturated or aromatic heterocycle having 1 to 2 N and/or O atoms,
and their pharmaceutically tolerated salts, solvates and stereoisomers.

11. Compounds according to claim 1:
a) 1-(3-amidinobenzyl)-3-(2′-sulfamoylbiphenyl-4-yl)-1-propylurea;
b) 1-(3-amidinobenzyl)-3-(2′-methylsulfonyl)-1-propylurea;
c) 1-(3-amidinobenzyl)-3-(2′-sulfamoylbiphenyl-4-yl)-1-phenyl-harnstoff;
d) 1-(3-amidinobenzyl)-3-(2′-methylsulfonyl)-1-phenylurea;
and their pharmaceutically tolerated salts and solvates.

12. Process for the preparation of compounds of the formula I according to claim 1 and their salts, characterised in that
a) they are liberated from one of their functional derivatives by treatment with a solvolysing and/or hydrogenolysing agent by
i) liberating an amidino group from their oxadiazole derivative or oxazolidinone derivative by hydrogenolysis or solvolysis,
ii) replacing a conventional amino-protecting group with hydrogen by treatment with a solvolysing or hydrogenolysing agent or liberating an amino group protected by a conventional protecting group,
and/or
b) converting a base or acid of the formula I into one of its salts.

13. Compounds of the formula I according to claims 1 to 11 and their physiologically acceptable salts and solvates as medicaments.

14. Medicaments according to claim 13 as inhibitors of coagulation factor Xa.

15. Medicaments according to claim 13 as inhibitors of coagulation factor VIIa.

16. Medicaments according to claim 13, 14 or 15 for the treatment of thrombosis, myocardial infarction, arteriosclerosis, inflammation, apoplexia, angina pectoris, restenosis after angioplasty, claudicatio intermittens, tumours, tumour diseases and/or tumour metastases.

17. Pharmaceutical preparation comprising at least one medicament according to one of claims 13 to 16 and optionally excipients and/or auxiliaries and optionally other active ingredients.

18. Use of compounds according to one of claims 1 to 11 and/or their physiologically acceptable salts and solvates for the preparation of a medicament for the treatment of thrombosis, myocardial infarction, arteriosclerosis, inflammation, apoplexia, angina pectoris, restenosis after angioplasty, claudicatio intermittens, tumours, tumour diseases and/or tumour metastases.
